(19) United States
(12) Patent Application Publication
(10) Pub. No.: US 2023/0103552 A1
(43) Pub. Date: Apr. 6, 2023

(54) SYSTEMS AND PHARMACEUTICAL COMPOSITIONS FOR TREATMENT BY DIRECT INJECTION OF A TARGETED POPULATION OF CELLS

(71) Applicant: Privo Technologies, Inc., Peabody, MA (US)

(72) Inventors: Manijeh Nazari Goldberg, Newburyport, MA (US); Aaron M. Manzi, Haverhill, MA (US); Eric Goldberg, Newburyport, MA (US); Michael K. Harris, Chestnut Hill, MA (US)

(21) Appl. No.: 17/758,205

(22) PCT Filed: Jan. 3, 2021
(86) PCT No.: PCT/US2021/012015

§ 371 Date: Jun. 29, 2022

Related U.S. Application Data
(60) Provisional application No. 62/956,795, filed on Jan. 3, 2020.

(51) Int. Cl. A61K 9/50; A61K 9/06; A61K 47/36; A61K 47/02; A61K 9/00; A61K 9/19; C07K 16/22; A61P 35/00; A61K 38/20; A61M 5/178

(52) U.S. Cl. CPC A61K 9/5036; A61K 9/06; A61K 47/36; A61K 47/02; A61K 9/0019; A61K 9/19; C07K 16/22; A61P 35/00; A61K 38/208; A61M 5/178; C07K 2317/24; A61K 2039/505

(57) ABSTRACT

Systems and methods are provided for delivering a therapeutic treatment to a targeted population of cells of a subject, including but not limited to tumors, eyeballs, pancreatic tissue, liver tissue, and lung tissue. The system includes an injectable aqueous solution in a vial enclosed with a septum. The solution includes particles containing a therapeutic agent and having a coating around the therapeutic agent, the coating including chitosan so as to provide controlled release of the agent from the particles. The solution further includes chitosan polymer in the form of a polymer gel matrix, further providing controlled release of the particles from the aqueous gel environment. Also provided are methods of manufacturing a lyophilized powder disposed within a vial containing chitosan polymer and chitosan coated particles, the powder forming the above-described injectable aqueous solution of particles and chitosan gel upon mixing with water.

### CROSS REFERENCE TO RELATED APPLICATIONS

The present application claims the benefit of U.S. provisional application No. 62/956,795 filed on Jan. 3, 2020, the disclosure of which is incorporated herein by reference in its entirety.

### TECHNICAL FIELD

The present invention relates to therapeutic compositions for injection into targeted cell populations for the treatment of diseases and tumors and more particularly to injectable aqueous solutions of chitosan gels and chitosan particles containing therapeutic agents, as well as methods of preparing and using such compositions.

### BACKGROUND ART

Tumors in humans are often treated through surgical excision. Tumor treatment is usually an urgent matter, especially the treatment of solid malignant tumors. These tumors include myeloid sarcomata, round-celled sarcomata, melanotic sarcoma, spindle-cell sarcoma, and papillomata. Other types of solid tumors are well known to those skilled in the medical arts.

Frequently, tumors are not fully resectable, and these solid tumors are considered inoperable. Inoperable solid tumors can be categorized as such due to their location, or their size. Chemotherapy is often used in the treatment of solid tumors to shrink their size, thereby rendering them operable.

Chemotherapy can be administered by three different routes: (1) systemic intravenous (IV), (2) intra-arterial, and (3), intratumoral. Systemic preoperative I.V. therapy has been found to be effective in reducing or shrinking a solid tumor. Ferriere, J. P. et al. (1998) Primary chemotherapy in breast cancer: correlation between tumor response and patient outcome, American journal of clinical oncology, 21(2), 117-120. Moreover, the I.V. route gives concurrent treatment to the entire organism so that metastatic cells (or micrometastases) are treated throughout the body. However, one of the major issues is delivering enough of the antitumor agent to the targeted location. Systemic chemotherapy can cause severe side effects that can be dose limiting and intolerable for the patient. These side effects reduce the use of particularly powerful and potent drugs. According to the literature, most drugs are administered systemically at the limit of tolerable side effects (MTD-maximum tolerated dose), at doses which do not provide optimum efficacy.

This limitation to the MTD not only affects success of the treatment, but also may have the counterproductive result of forming a more resistant tumor. It is assumed that there are several populations of the same type of tumor cell within a specific solid tumor that differ from one another by their ability to resist a chemotherapeutic agent at a particular dose level. Kinsella, A. R., Smith, D., & Pickard, M. (1997) Resistance to chemotherapeutic antimetabolites: a function of salvage pathway involvement and cellular response to DNA damage, British journal of cancer, 75(7), 935). The MTD may be a dose level which is capable of killing most, but not all, of the cells in the particular tumor. As a result, not only do residual amounts of more resistant cancer cells remain but also, due to extensive proliferation, those more resistant cells will dominate most of the tumor and will provide a more difficult challenge for treating that tumor chemically in the future. Another obstacle is that many antineoplastic drugs may be phase sensitive. That is, they interact with the cells only when the cells are in a particular stage of the cell cycle. Other cells, not in the sensitive stage at the time of dosing, are spared. I.V. dosing, being of relatively short duration, may miss the sensitive phase of the tumor cells even with high dose intensity. Treatment of many tumors could benefit from a lower dose, higher frequency or continuous dosing schedule both in efficacy and in lowering adverse event intensity.

Intratumoral injection is a promising alternative technique for chemotherapy and, at least conceptually, should present the most successful approach. In this method, the antineoplastic drug is administered directly to the tumor, thus achieving high local concentrations and avoiding systemic side effects. This method also provides an almost infinite flexibility in dosage.

In spite of these advantages, intratumoral chemotherapy has not been particularly effective. It has been proposed that this lack of efficacy reflects one or more of the following factors:

The density of the tumor cells in the tumor is very high, thus preventing drug penetration through the cells when it is not via the blood vessels.

The interstitial fluid pressure is high, preventing migration of the drug into the interstitial fluid.

The high density of cells and blood vessels causes the blood vessels themselves to constrict. See Jain, R. K. (1999) Transport of molecules, particles, and cells in solid tumors, Annual review of biomedical engineering, 1(1), 241-263.

Dosing protocols to alleviate these problems by inducing apoptosis in the tumor have been proposed. See, e.g., M. Flashner-Barak, U.S. patent application Ser. No. 2002/0041888 A1, Ser. No. 09/829,621.

Other possible reasons for failure of intratumoral dosing include non-homogeneous spread of the drug throughout the tumor and the lack of an effective dose for a long enough period to treat the cells when they enter their sensitive phase in the cycle. The problem in intratumoral chemotherapy then reduces to maintaining a high enough concentration of a chemotherapeutic agent over a long enough time period, spread throughout the tumor, in order to achieve these goals. Intratumoral injections have been carried out using gels, pastes and microparticles.

Chitosan is a nontoxic (LD50>16 g/kg), biodegradable, natural polysaccharide derived from the exoskeletons of crustaceans. The source of chitosan is chitin, a natural biopolymer most abundant in exoskeletons of crustaceans and insect cuticles, cell walls of fungi, shells of mollusks, etc. Chitin consists of 2-acetamido-2-deoxy-β-D-glucose monomers (N-acetyl glucosamine units) linked through β(1→4) linkages and chitosan is a polymer of deacetyl α-(1, 4) glucosamine units that can typically be obtained by deacetylation of chitin with NaOH after demineralization and deproteinization of the crustacean shells or exoskeletons. Chitosan is a multi-functional material with good biocompatibility, no immunogenicity and no skin irritation. In 2001, it was approved by Food and Drug Administration of the United States (FDA) as a GRAS (Generally Recognized as Safe) substance. Chitosan is a widely used biomaterial with an established safety profile in humans. It is used as a pharmaceutical excipient, a weight-loss supplement, and an experimental mucosal adjuvant, and in an FDA-approved hemostatic dressing. High-molecular-weight chitosan (>100 kDa), by virtue of its long polymer chains, forms highly viscous solutions in mild aqueous solvents. Viscous solutions have been widely used to control release of drugs and macromolecules in vivo, as they hinder the diffusion and dissemination of these molecules following injection. Baldrick, P. (2010) The safety of chitosan as a pharmaceutical excipient, Regulatory toxicology and pharmacology, 56(3), 290-299.

Platinum-based drugs, such as cisplatin (cis-diamminedichloroplatinum-II), are among the most widely used chemotherapeutic agents and have shown efficacy against various solid neoplasms outside the central nervous system, including testicular, ovarian, breast, colorectal, lung, head and neck tumors. Systemically delivered cisplatin penetrates poorly into normal brain tissue due to the blood-brain barrier (BBB) with less than 5% of the plasma concentration detected in the brain after intravenous delivery. However, the neo-vasculature in tumors is more permeable than the intact BBB, and therapeutic cisplatin levels have been detected in primary and secondary brain tumors and to a lesser extent in the edematous brain adjacent to tumor after systemic delivery. Pérez, J. et al. (2019) The effect of locally delivered cisplatin is dependent on an intact immune function in an experimental glioma model, Scientific reports, 9(1), 5632.

Cytokines, small proteins released by immune cells, allow immune cells to communicate with each other. Cytokines have been investigated for some time as a potential cancer treatment. However, despite their known potency and potential for use alongside other immunotherapies, cytokines have yet to be successfully developed into an effective cancer therapy. This failure likely reflects the high toxicity of cytokines to both healthy tissue and tumors alike, making them unsuitable for use in treatments administered to the entire body.

Injecting cytokines directly into tumors could provide a method of confining their toxic effects to the tumor and sparing healthy tissue, but previous attempts to do so have resulted in the proteins leaking out of the cancerous tissue and into the body's circulation within minutes.

Cytokines are a broad and loose category of small proteins (˜5-20 kDa) that are important in cell signaling. Cytokines are peptides, and cannot cross the lipid bilayer of cells to enter the cytoplasm. Cytokines are involved in autocrine signaling, paracrine signaling and endocrine signaling as immunomodulating agents. Their definite distinction from hormones is still part of ongoing research.

Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. Aznar, M. A. et al. (2017) Intratumoral delivery of immunotherapy—act locally, think globally, The Journal of Immunology, 198(1), 31-39.

Cytokines act through receptors, and are especially important in the immune system, where they modulate the balance between humoral and cell-based immune responses, and they regulate the maturation, growth, and responsiveness of particular cell populations. Some cytokines enhance or inhibit the action of other cytokines in complex ways.

Cytokines include but are not limited to granulocyte colony-stimulating factor (G-CSF), interferons, interleukins including IL-2, IL-7, IL-12, and various chemokines.

Other immunomodulatory agents are also being investigated, including imiquimod, cellular membrane fractions from bacteria being used on patients and others, synthetic cytosine phosphate-guanosine (CpG), oligodeoxynucleotides and glucans.

Ocular vascular diseases are among the leading causes of visual impairment and blindness worldwide. Intravitreal injection of anti-vascular endothelial growth factor (anti-VEGF) agents has revolutionized the treatment of common retinal diseases, including neovascular age-related macular degeneration (AMD), diabetic retinopathy, and retinal vein occlusions (RVOs). Moreover, promising results were reported with intravitreal injection of anti-VEGF agents for other ocular diseases, such as neovascular glaucoma, retinopathy of prematurity (ROP), and intraocular tumors.

Age-related macular degeneration (AMD) is a well-characterized and extensively studied disease. It is currently considered the leading cause of visual disability among patients over 60 years. The hallmark of early AMD is the formation of drusen, pigmentary changes at the macula, and mild to moderate vision loss. There are two forms of AMD: the “dry” and the “wet” form that is less frequent but is responsible for 90% of acute blindness due to AMD. Risk factors have been associated with AMD progression, and they are relevant to understand how AMD develops: (1) advanced age and the exposition to environmental factors induce high levels of oxidative stress damaging the macula and (2) this damage, which causes inflammation induces a vicious cycle, eventually causing central vision loss.

There is neither a cure nor treatment to prevent AMD. However, there are some treatments available for the wet form of AMD. The treatment of the wet form had a major breakthrough due to the introduction of antiangiogenic drugs; the functional prognosis changed from almost-certain blindness to more than 90% chance of three-line visual improvement after two years of treatment. Nevertheless, even after this progress, therapy is far from perfect and there is still ample room for improvement. Hernandez-Zimbrón, L. F. et al. (2018) Age-Related Macular Degeneration: New Paradigms for Treatment and Management of AMD, Oxidative medicine and cellular longevity, 2018, 8374647.

Reducing the treatment burden associated with regular anti-VEGF intravitreal injections is a priority. Neovascular AMD and diabetic retinopathy are chronic, relapsing disorders. Patients may require tens of injections over many years of treatment. Compliance with such a demanding regimen is challenging. Current promising approaches include (a) new hardware to deliver anti-VEGF medications (b) new pharmaceuticals with longer durability of biological effect (c) novel formulations of anti-VEGF agents for sustained release and (d) gene therapy. Puliafito, C. A. et al. (2019) Looking ahead in retinal disease management: highlights of the 2019 angiogenesis, exudation and degeneration symposium. International journal of retina and vitreous, 5(1), 22.

Currently, several anti-VEGF drugs, including pegaptanib, ranibizumab, bevacizumab, and aflibercept, are available. Although well-designed randomized clinical trials have shown the efficacy of these agents in visual improvement in various retinal diseases, each intravitreal injection poses the risk of post-injection- and drug-class-associated adverse events. The repeated and long-term injections that are commonly needed may increase the chance of ocular and systemic complications. Falavarjani, K. G. et al. (2013) Adverse events and complications associated with intravitreal injection of anti-VEGF agents: a review of literature, Eye, 27(7), 787.

In particular, the most common treatment for AMD is intravitreal bevacizumab injection. Injections are administered every three to four weeks and can be a source of procedural risk and inconvenience for the patient.

Targeted delivery of drugs to other tissues, including but not limited to pancreatic tissue, lung tissue and liver tissue, localizes treatment to these tissues, and has potential benefits for the treatment of diseases specific to these tissues, including pancreatitis, diabetes, brain cancer, lung cancer, and hepatitis.

### SUMMARY OF THE EMBODIMENTS

In one embodiment of the invention, there is provided a composition formulated for delivery by injection into a targeted population of cells of subject. In this embodiment, the composition includes an aqueous solution including a chitosan gel and a plurality of particles, the particles containing a therapeutic agent and having a coating around the therapeutic agent, the coating including chitosan so as to provide controlled release of the agent from the particles.

One embodiment of the invention provides a system for delivering a therapeutic treatment to a targeted population of cells of a subject. In this embodiment, the system includes a vial enclosed with a septum that is penetrable by a needle of a syringe to be used for administration of the therapeutic treatment. In this embodiment, a therapeutic composition is disposed in the vial, the therapeutic composition provided for use in administration of the therapeutic treatment and comprising an aqueous solution including a chitosan gel and a plurality of particles embedded in the gel, the gel having a viscosity rendering it suitable for administration by injection. The particles in this embodiment contain a therapeutic agent and have a coating around the therapeutic agent, the coating including chitosan so as to provide controlled release of the agent from the particles.

In a further related embodiment, the aqueous solution further includes a compound selected from a group consisting of a hydration promotor, a particle adhesion inhibitor, a particle aggregation inhibitor, and combinations thereof.

The hydration promotor is selected from the group consisting of ethylene glycol, propylene glycol, beta-propylene glycol, glycerol and combinations thereof.

The particle adhesion inhibitor is selected from the group consisting of HPMC, poloxamer, and combinations thereof.

The particle aggregation inhibitor is selected from the group consisting of monosaccharides, disaccharides, sugar alcohols, chlorinated monosaccharides, chlorinated disaccharides, and combinations thereof.

Alternatively or in addition, the composition further includes sodium tripolyphosphate.

In some embodiments, the particles are microparticles having an average diameter between 200 nm and 2000 nm. As a further option, the microparticles have an average diameter between 500 nm and 2000 nm.

Optionally, the aqueous solution further includes a free quantity of the therapeutic agent, not coated with chitosan, wherein the free quantity of therapeutic agent comprises between about 20% to about 80% of a total quantity by weight of therapeutic agent in the aqueous solution.

Also optionally, the therapeutic agent in the particles is an immunotherapeutic. As a further option, the therapeutic agent is selected from the group consisting of an antibody, a cytokine, a small molecule immunotherapeutic, and combinations thereof.

In a further related embodiment, the therapeutic agent is a chemotherapeutic.

Optionally, the particles are in direct physical contact with the chitosan gel.

In another embodiment, the invention provides a method for treatment of a targeted population of cells of a subject. This method includes obtaining a system as described at the beginning of this section, loading the aqueous solution into the syringe, and injecting the aqueous solution into the targeted population of cells by means of the syringe.

In a related embodiment of the method, the targeted population of cells includes a tumor. In another related embodiment, the targeted population of cells is tissue in an organ. Optionally, the organ is selected from the group consisting of eye, lung, pancreas, liver, kidney, brain, heart, thyroid, and pituitary.

In another embodiment, a system is provided for delivering a therapeutic treatment to a targeted population of cells of a subject, the system including a vial enclosed with a septum that is penetrable by a needle of a syringe to be used for administration of the therapeutic treatment. In this embodiment, a therapeutic composition is disposed in the vial, the therapeutic composition provided for use in administration of the therapeutic treatment and including a lyophilized precursor formulated so that upon mixing with water, it dissolves to provide an aqueous solution including chitosan gel and a plurality of particles embedded in the gel, the gel having a viscosity rendering it suitable for administration by injection. The particles in this embodiment contain a therapeutic agent and have a coating around the therapeutic agent, the coating including chitosan so as to provide controlled release of the therapeutic agent from the particles.

Another embodiment provides a lyophilization method for providing a system for delivering a therapeutic treatment to a targeted population of cells. This method includes: (1) forming an aqueous solution including a chitosan gel and a plurality of particles, the particles containing a therapeutic agent and having a coating around the therapeutic agent, the coating including chitosan so as to provide controlled release of the agent from the particles, (2) freezing the aqueous solution in a bath containing an aqueous alcoholic solution at a temperature above the freezing temperature of the aqueous alcoholic solution and at most −80° C., to form a frozen layer precursor, (3) drying the frozen layer precursor, to form anhydrous powder embedded with particles, (4) including the anhydrous powder in a vial enclosed with a septum that is penetrable by a needle of a syringe to be used for administration of the therapeutic treatment, and (5) adding water to the vial to dissolve the anhydrous powder.

Optionally, the aqueous solution in the lyophilization method further includes a hydration promoter, a particle adhesion inhibitor, and a particle aggregation inhibitor.

In a further related embodiment of the lyophilization method, the hydration promotor is selected from the group consisting of ethylene glycol, propylene glycol, beta-propylene glycol, glycerol and combinations thereof. Also optionally, the particle adhesion inhibitor comprises HPMC, poloxamer, and combinations thereof. In a related embodiment, the particle aggregation inhibitor is selected from the group consisting of monosaccharides, disaccharides, sugar alcohols, chlorinated monosaccharides, chlorinated disaccharides, and combinations thereof. Optionally, the composition further includes sodium tripolyphosphate. Also optionally, the particles are microparticles having an average diameter between 200 nm and 2000 nm. As a further option, the particles are microparticles having an average diameter between 500 nm and 2000 nm.

Some embodiments of the invention, a particle-based composition is formulated for delivery by intravitreal injection for treating an ocular condition.

Optionally, the ocular disease is age-related macular degeneration (AMD).

According to some embodiments, the therapeutic agent for intravitreal injection is selected from the group consisting of an antibody, a cytokine, a small molecule immunotherapeutic, a chemotherapeutic, an aptamer, and combinations thereof. In some embodiments the therapeutic agent for intravitreal injection is bevacizumab.

### BRIEF DESCRIPTION OF THE DRAWINGS

The foregoing features of embodiments will be more readily understood by reference to the following detailed description, taken with reference to the accompanying drawings, in which:

FIG. 1A provides an injectable chitosan formulation according to the instant invention (PRV311), frozen at −80° C. and stored properly. Notably, the formulation provides a clear aqueous solution. (Left) versus PRV311 at room temperature (Middle), and PRV111 (Right). PRV111 is a similar product to PRV311 but is frozen more quickly in liquid nitrogen (−196° C.) rather than in a freezer at −80° C. when it is made.

FIG. 1B shows for comparison an injectable chitosan formulation according to the instant invention (PRV311), frozen at −80° C. and stored at room temperature for five days.

FIG. 1C shows for comparison an injectable chitosan formulation according to the instant invention (PRV311), frozen in liquid nitrogen at −196° C. and stored at room temperature for five days.

FIG. 2 is an FTIR Spectrum of injectable chitosan powder comprising cisplatin internal standard, with peaks at 1400 and 1560 cm⁻¹.

FIG. 3 is a photograph of the matrix (2× zoom) when frozen in liquid nitrogen (−196° C.) and subsequently lyophilized. Note the multi-layered, dense, fabric-like structure.

FIG. 4 is a photograph of the matrix (2×zoom) when frozen in a −80° C. chest freezer and subsequently lyophilized. Note the more porous, uniform, single-layer polymer fibers.

FIG. 5 is a release profile of drug from the microparticles in media of varying pH levels. Powders were reconstituted in their respective media and placed inside of dialysis bags under stirring for 72 hours. Samples were taken and the percent release is shown, with microparticles at pH 6 (circles) releasing faster due to more rapid degradation, and those at pH 3 (triangles) released at a slower rate due to higher particle stability. Free cisplatin solution (squares) was used as a control.

FIG. 6 provides a graph of change in Mice tumor volume vs time involving different treatments. The black curve corresponds to a control untreated tumor, the gray curve corresponds to intratumoreal injection with placebo particles containing no drug, the green curve corresponds to intravenous injection with drug, the red curve corresponds to intratumoreal injection of free drug, and the blue curve corresponds to injection with drug-encapsulated hydrogel PRV311.

FIG. 7 is a cross-section of lamb tissue following an injection with PRV311, a chitosan formulation in accordance with an embodiment of the present invention, viewed by fluorescent microscopy. Here, the drug is labeled with fluorescein isothiocyanate (FITC), and appears green under the microscope.

FIG. 8 is a graph showing FITC labeled drug concentration within pig tongue tissue as a function of tissue depth.

FIG. 9 is a photograph showing intratumoral injection using an embodiment of the present invention and the local distribution of drug labelled with FITC.

FIG. 10 is a photograph showing penetration into a cow brain of an injectable in accordance with an embodiment of the present invention.

FIG. 11 is a drawing illustrating, greatly enlarged, an embodiment of the present invention as constituted for injection.

FIG. 12 is a drawing illustrating, greatly enlarged, how the injectable solution PRV311, in accordance with an embodiment of the present invention, builds a polymeric web within and around the tumor.

FIG. 13A and FIG. 13B are photomicrographs illustrating PRV311, in accordance with embodiments of the present invention.

FIG. 14 is a photograph of showing some of more than 80 formulations, evaluated by the inventors, in the course of developing a formulation suitable for reconstitution for clinical use within few seconds in accordance with embodiments of the present invention. Notably, the solutions are clear.

FIG. 15A, FIG. 15B, and FIG. 15C. show an injection of a PRV311 formulation with fluorescently labeled drug into sheep's eye, demonstrating complete corneal coverage and penetration.

FIG. 16 shows injection with separate fluorescent labeling of polymer (red) and drug (green), illustrating how both bolus and controlled delivery can be effected.

FIG. 17 shows injection of fluorescently labeled PRV311 into lung tissue, showing a high local concentration of drug for over 24 hours post injection.

FIG. 18 shows the injection of PRV311 into sheep's liver, again showing a high local concentration of drug.

FIG. 19 shows a side view of injection of PRV311 into sheep's liver, showing that tissue type impacts the drug distribution pattern.

FIG. 20 shows local injection of PRV311 into sheep pancreas, showing a high local drug concentration and that tissue type impacts the localized drug distribution pattern.

### DETAILED DESCRIPTION OF SPECIFIC EMBODIMENTS

Definitions. As used in this description and the accompanying claims, the following terms shall have the meanings indicated, unless the context otherwise requires:

A “subject” includes a vertebrate, such as a mammal, and, further, such as a human being.

A “polymer” is a molecule having at least 100 units of a monomer.

A polymeric “matrix” is a three-dimensional web of polymer molecules, the web being chosen from the group consisting of non-covalently entangled, ionically cross-linked, covalently cross-linked, and combinations thereof.

A “gel” is a solution phase of a polymeric matrix that is swollen in solvent, while retaining entanglements and cross-linkings.

“Microparticles” are sets of particles having an average diameter of about 200 nm to about 2000 nm. “Nanoparticles” are sets of particles having an average diameter of at least 1 nm to about 200 nm.

A “particle diameter” or “particle size” is the length of the longest straight axis between two points on the surface of the particle.

A “pure chitosan” is a chitosan that is not a salt of chitosan.

An “unmodified chitosan” is a chitosan that is not chemically modified by the addition of functional groups, or by linkage to a carrier.

An “unmodified therapeutic agent” is a therapeutic agent that is not chemically modified by the addition of functional groups, or by linkage to a carrier.

An “immunotherapeutic” is a therapeutic agent that modulates the immune response. An immunotherapeutic may be a biological or a small molecule drug.

A “chemotherapeutic” is a therapeutic agent that is a small-molecule drug.

An “aptamer” is a nucleic acid or modified nucleic acid that has been selected by means of in vitro selection methods for binding to a biological target. A notable example of an “aptamer” is the drug pegaptanib (trade name Macugen®) which binds VEGF and is used for the treatment of wet macular degeneration.

A “microparticle adhesion inhibitor” is an additive that lowers the attractive forces between a polymeric matrix and particles embedded therein. As a result, the particles can move through the matrix at a faster rate than in the absence of the adhesion inhibitor.

A “microparticle aggregation inhibitor” is an additive that lowers the tendency of particles embedded in a matrix to aggregate when the matrix is subjected to freezing. As a result, the particles are less likely to suffer from damage or destruction when the freezing takes place.

A “mucoadhesive” material is characterized as having the ability to adhere to mucosal membranes in the human body.

A polymeric matrix is “porous” when a fraction of its volume is void space. In some instances, the void space is accessible from the outer surface of the matrix, so that items present in the void space, such as microparticles, may migrate to and from the outer surface.

A “void” space in a polymeric matrix is space that is not occupied by polymer and allows the movement of microparticles and small molecules through the space.

“Mucosal tissue” is tissue having an associated mucosa. In particular, mucosal tissue includes the mucosa and also tissue underlying the mucosa.

A “site in mucosal tissue”, where, for example, a cancerous tumor is present may involve not only the mucosa but also tissue underlying the mucosa.

“Polydispersity index” (PDI) or simply, “dispersity” is a measure of the heterogeneity of sizes of a set of particles, for example microparticles in a mixture.

“Zeta potential” (ZP) is a measure of the overall charge that a particle acquires in a particular medium. The ZP may be measured on a Zetasizer Nano instrument.

“Permeation” is the ability to pass through or penetrate, a mucosa, its underlying tissue, or both. “Biocompatible” refers to the ability of a biomaterial to perform its desired function with respect to a medical therapy, without eliciting any significant undesirable local or systemic effects in the recipient or beneficiary of that therapy, but generating the most appropriate beneficial cellular or tissue response in that specific situation, and optimizing the clinically relevant performance of that therapy.

“HPMC” refers to hydroxypropyl methylcellulose, also known as hypromellose.

“Biodegradable” refers to a property of the materials that is capable of being broken down especially into innocuous products by the action of living things.

“Kilo count per second” (Kcps)”, mean count rate (in kilo counts per second (kcps)). For example, the threshold may be set such that when the count rate of the sample is lower than 100, the measurement should be aborted, meaning the concentration of the sample is too low for measurements. A sample with suitable Kcps can be considered a stable sample with idea concentration for measurement.

“Mesh” refers to a polymeric matrix, adherent to the treated area, which contains elements incorporated within it to be released from the mesh when it is applied to the treated area.

A “system for delivery of a therapeutic agent based on a polymeric matrix and microparticles” may also be referred to as an “agent delivery device” or as a “delivery patch”.

Unless otherwise specified, the term “wt %” refers to the amount of a component of a system for delivery of a therapeutic agent, as expressed in percentage by weight.

Unless otherwise specified, the “molar mass” of a polymer is intended to mean the number average molar mass of the polymer molecules.

Cancer can develop in any tissue of any organ at any age. Once an unequivocal diagnosis of cancer is made, treatment decisions become paramount. Though no single treatment approach is applicable to all cancers, successful therapies must be focused on both the primary tumor and its metastases. Historically, local and regional therapy, such as surgery or radiation, have been used in cancer treatment, along with systemic therapy, e.g., chemotherapy drugs. Despite some success, conventional treatments are not effective to the degree desired, and the search has continued for more efficacious therapies. There is clearly a significant unmet need for more efficient cancer therapies

One of the major uses for embodiments of the present invention is for intratumoral injections of chemotherapy and immunotherapy, with the data that demonstrates the best way to maintain a high concentration of the drug in the tumor and also drain some of the drug to the lymph nodes to ensure the most effective way to treat the tumor in a local and regional manner.

Intratumoral injections can be considered for any tumor where the primary lesion or its metastases are accessible either percutaneously via direct injection or via specific procedures such as colonoscopy, cystoscopy, bronchoscopy, thoracoscopy, coelioscopy, or even surgery.

There is now a plethora of agents being investigated for their role in intratumoral therapy, including immune receptor agonists (such as Toll-like receptor (TLR) agonists and stimulator of interferon gene (STING) agonists, ICT mAbs, wild-type and genetically-modified oncolytic agents (such as viruses and peptides), cytokines and immune cells directed at a variety of potential targets). Thus, to support the clinical development of human intratumoral strategies, the inventors developed an injectable system for local delivery and retention of these agents.

Furthermore, direct injection into the tumor reduces systemic exposure, off-target toxicities, and the amounts of drug used while inducing stronger antitumor activity in the injected tumor lesion and in distant noninjected tumor lesions.

Systemic immunotherapy and systemic chemotherapy are often used but they expose the patient's entire body to the drugs' toxic side effects. Systemic administration is dose limiting due to exposure within the blood stream and other organs, as precautions must be taken in consideration of the safety of this systemic exposure. Systemic delivery often results in damaging side effects from toxic drugs reacting with the body. These include neurotoxicity, nephrotoxicity, kidney failure, hair loss, nausea and mucositis. As an alternative to surgery, chemotherapy in addition to radiation are also used as methods to treat anal tumors. The current standard of care uses initial concurrent combination of chemotherapy and radiation for patients with anal canal squamous cell carcinoma, even with small, local tumors. When chemotherapy is used, temporary central venous catheters or peripherally inserted central catheters may be used on an individual. Side effects from treatment include those typical to systemic chemotherapy. These include nausea, hair loss, kidney damage, low blood cell count, mouth sores and a compromised immune system. Since chemotherapy is currently delivered systemically throughout the body, there are dose limiting factors

A drug's therapeutic advantage may be increased by maximizing its efficacy and/or reducing its side effects. The basis for the development of a regional cancer drug therapy is the achievement of effective target tissue concentrations while minimizing systemic distribution and therefore toxicity. Examples of existing, clinically used, regional chemotherapies include intra-arterial infusion for liver and kidney neoplasms, limb perfusions for melanoma and sarcomas, intrathecal administration for CNS neoplasms, and intraperitoneal administration for intra-abdominal neoplasms. More recently, a direct intratumoral injection of pure ethanol for primary hepatomas has been developed. One major drawback associated with most cytotoxic chemotherapeutic agents is the fact that they are strong vesicants, and thus are not ideal candidates for intratumoral administration unless the delivery technology can maintain the drug locally to the tumor and not allow for leakage to the healthy tissue. Embodiments of the invention use a combination of polymeric drug loaded chitosan particles and combinations of polymers to ensure drug retention in the tumor and reduced side effects.

Intratumoral immunotherapy is a therapeutic strategy which aims to use the tumor as its own vaccine. Upon direct injections into the tumor, a high concentration of immunostimulatory products can be achieved in situ, while using small amounts of drugs. Local delivery of immunotherapies allows multiple combination therapies, while preventing significant systemic exposure and off-target toxicities. Despite being uncertain of the dominant epitopes of a given cancer, one can therefore trigger an immune response against the relevant neo-antigens or tumor-associated antigens without the need for their characterization. Such immune stimulation can induce a strong priming of the cancer immunity locally while generating systemic (abscopal) tumor responses, thanks to the circulation of properly activated antitumor immune cells. While addressing many of the current limitations of cancer immunotherapy development, intratumoral immunotherapy also offers a unique opportunity to better understand the dynamics of cancer immunity by allowing sequential and multifocal biopsies at every tumor injection. Marabelle, A. et al. (2018) Starting the fight in the tumor: expert recommendations for the development of human intratumoral immunotherapy (HIT-IT), Annals of oncology: official journal of the European Society for Medical Oncology, 29(11), 2163-2174.

All five classes of immunotherapy face delivery challenges. Checkpoint inhibitors, cytokines, and agonistic antibodies have similar delivery challenges. The success of these therapies relies on their interaction with the targeted protein. A major limitation of their use is that they produce substantial autoimmunity, leading to adverse effects that limit the allowable administered doses. For this reason, a central goal in the development of delivery technologies for these therapies is to enable targeted and controlled release so that the therapies are primarily active in the desired cell types, thereby minimizing off-target effects.

The microenvironment in many solid tumors is a challenge to the broad implementation of all the immunotherapy classes discussed here. For example, the microenvironment of solid tumors can be categorized as either immunologically ‘hot’ (high immunogenicity) or ‘cold’ (low immunogenicity), which have either high or low levels of cytotoxic lymphocyte infiltration within the tumor space, respectively. This key difference in the composition of the microenvironment suggests that tumors with high immunogenicity exhibit stronger responses to checkpoint inhibitors than do tumors with low immunogenicity.

Delivery technologies can be exploited to modulate immunogenicity in cold tumors. In addition, because delivery platforms can also reduce the systemic toxicity of immunotherapies by limiting drug exposure to particular tissues, they can be used to deliver combinations of therapeutics that would otherwise be too toxic to administer to patients.

Local delivery of immunotherapies using embodiments of the present invention allows multiple combination therapies, while preventing significant systemic exposure and off-target toxicities. Despite being uncertain of the dominant epitopes of a given cancer, one can trigger an immune response against the relevant neo-antigens or tumor-associated antigens without the need for their characterization. Such immune stimulation can induce a strong priming of the cancer immunity locally while generating systemic (abscopal) tumor responses, thanks to the circulation of properly activated antitumor immune cells.

In accordance with embodiments of the present invention, tumors can be injected with compositions including combinations of one or more of immunotherapeutic particles and chemotherapeutic particles. Chemotherapeutic particles may contain chemotherapeutics including but not limited to cisplatin and oxaliplatin, which have been shown to activate dendritic cells and induce immune activity in tumors in addition to causing DNA-damaging effects in tumor cells.

Embodiments of the present invention when delivering chemotherapy can cause immunologically cold tumors to become hot and therefore make them susceptible to immunotherapy. The tumor-targeted immunotherapy particles and chemotherapy work synergistically to inhibit tumor growth and exhibit reduced toxicity compared to that of immunotherapy and chemotherapy alone, i.e. without using embodiments of the invention.

We have found that microparticles can enable combination treatment strategies to make tumors with low immunogenicity susceptible to immunotherapy. In addition to enabling combination treatment strategies, embodiments of the present invention can be designed to respond to the tumor microenvironment and increase penetration at those sites.

According to David Zaharoff et al., a chitosan mixture with the cytokine IL-12 has been effective in tumor regression in their mice experiments (Zaharoff, D. A., et al. (2010). Intratumoral immunotherapy of established solid tumors with chitosan/IL-12. J. Immunother., 33, 697). However the inventors' data shows that, in accordance with embodiments of the present invention, the combination of chitosan matrix chitosan loaded with IL12 particles has a very high retention time (>10 days) along with controlled release in the tumor in high concentrations. In various embodiments, injectable cytokines can be mixed in the clinic at the bedside within seconds for translational application, unlike the lab experiments done in Zaharoff s work.

IL-12 is a potent antitumor cytokine that exhibits significant clinical toxicities after systemic administration. Zaharoff hypothesized that intratumoral (i.t.) administration of IL-12 coformulated with the biodegradable polysaccharide chitosan could enhance the antitumor activity of IL-12 while limiting its systemic toxicity. Noninvasive imaging studies monitored local retention of IL-12, with and without chitosan coformulation, after i.t. injection. Antitumor efficacy of IL-12 alone and IL-12 coformulated with chitosan (chitosan/IL-12) was assessed in mice bearing established colorectal (MC32a) and pancreatic (Panc02) tumors. Additional studies involving depletion of immune cell subsets, tumor rechallenge, and CTL activity were designed to elucidate mechanisms of regression and tumor-specific immunity. Coformulation with chitosan increased local IL-12 retention from 1 to 2 days to 5 to 6 days. Weekly i.t. injections of IL-12 alone eradicated ≤10% of established MC32a and Panc02 tumors, while i.t. chitosan/IL-12 immunotherapy caused complete tumor regression in 80% to 100% of mice. Depletion of CD4+ or Gr-1+ cells had no impact on chitosan/IL-12-mediated tumor regression. However, CD8+ or NK cell depletion completely abrogated antitumor activity. I.t. chitosan/IL-12 immunotherapy generated systemic tumor-specific immunity, as >80% of mice cured with i.t. chitosan/IL-12 immunotherapy were at least partially protected from tumor rechallenge. Furthermore, CTLs from spleens of cured mice lysed MC32a and gp70 peptide-loaded targets. Chitosan/IL-12 immunotherapy increased local retention of IL-12 in the tumor microenvironment, eradicated established, aggressive murine tumors, and generated systemic tumor-specific protective immunity. Chitosan/IL-12 is a well-tolerated, effective immunotherapy with considerable potential for clinical translation.

In some embodiments, particle-based formulations are provided for the controlled delivery of drugs by intravitreal injection into the eye to treat ocular conditions, in particular age-related macular degeneration. In such embodiments, biocompatible polymers form a hydrogel matrix that forms a polymeric mesh that adheres to the epithelium of the eye where it implants drug particles into the tissue. The particles can degrade over a prolonged period of time, e.g. four months, to provide sustained release of the drug.

In accordance with one embodiment of the present invention, the inventors have developed a formulation for oral and injectable delivery of poorly water-soluble agents and polymers. The formulation has enabled converting a liquid nanocrystal dispersion into solid dosage form. The solid dosage form includes nanocrystals that can be readily reconstituted into their original size upon dissolution in water. Careful formulation is needed to optimize the freezing rate to decrease particle-particle aggregation. A critical freezing rate has been determined for drying nanocrystals. Freeze drying at a freezing rate near the critical value produces dry powders of bimodal particle size distribution after re-dispersion. In addition, drug nanocrystal concentration was found to significantly affect the critical freezing rate and therefore the re-dispersibility of dry powders. The concept of critical freezing rate is important for the development of solid dosage forms of liquid nanocrystal dispersions.

Embodiments of the present invention provide a formulation that can be shipped in powder form and can be rapidly, uniformly and consistently dissolved in sterile water for intratumoral injection at the bedside of patients.

Embodiments of the present invention provide systems for delivery of a therapeutic agent to various tissues, and in particular to cancerous tumors. Various embodiments include chitosan and a plurality of particles embedded within the matrix.

Once a working formulation of an embodiment of the present invention was developed, chitosan microparticles were synthesized at room temperature using ionic gelation with sodium tripolyphosphate as a cross-linker. A separate formulation of polymeric matrix containing particle adhesion inhibitors, particle aggregation inhibitor, and hydration promoters were added to the microparticle solution. Microparticle and matrix solutions were dispensed into vials, transferred to a −80° C. freezer, and allowed to freeze overnight. Then, the vials were lyophilized for 72 hours.

Some embodiments of the invention to produce injectable chitosan powder comprising cisplatin are based on the following protocol:
1. Chitosan Powder is added to Acetic acid solution (0.186 w/w %), stirred to dissolution.
2. In a separate container, cisplatin (0.15 w/w %) is added to a Sodium Tripolyphosphate and Saline solution. Cisplatin was dissolved by heating the solution to approximately 40° C. and stirring.
   a. All containers that have cisplatin in them were shielded from light exposure
3. and the contents of the Cisplatin-Sodium Tripolyphosphate solution were transferred to the Chitosan Solution
   a. Both solutions were gently stirred throughout this step.
   b. This step here produces microparticles. Once steady state is achieved, particle size/charge is gathered.
4. A sucralose solution (25 w/w %) in water was prepared and set aside for later use.
5. In a separate screw-top bottle, Chitosan powder is added to a dilute acetic acid solution (1.0 w/w %). Then, Hydroxypropyl methylcellulose (0.1 w/w %) is added to the chitosan-acetic acid solution. It is stirred for 30 minutes.
6. The sucralose solution was transferred to the Chitosan-Cisplatin microparticle solution. Then, the patch matrix solution was transferred.
7. The final solution was stirred for 5 minutes before 5 mL of solution was dispensed. The vials were frozen over approximately 2 hours in a −80° C. freezer.
8. Vials were placed in a lyophilizer for 6 days.

A sample Certificate of Analysis for injectable chitosan powder comprising cisplatin is shown in Table 1. FTIR characterization of the sample is shown in FIG. 2, where characteristic cisplatin peaks at 1400±30 cm⁻¹ and 1560±30 cm⁻¹ can be seen.

TABLE 1

| | | |
|---|---|---|
| Test | Acceptance Criteria | Results |

A sample Certificate of Analysis for injectable
chitosan powder comprising cisplatin:

| | | |
|---|---|---|
| Product Appearance | Light yellow powder | Conforms |
| Package Appearance | Sealed within a clear | Conforms |
| | Borosilicate Glass vial with | |
| | a black PTFE cap. The vial | |
| | and a desiccant pouch are | |
| | sealed within a mylar foil | |
| | pouch with no evidence of | |
| | leakage or breach. | |
| Identification & | Peak wavenumbers conform | Conforms |
| Characterization | to internal standard, with | |
| by FTIR | peaks at 1400 ± 30 cm⁻¹ and | |
| | 1560 ± 30 cm⁻¹ | |
| Assay by AAS | 85.0% to 115.0% of Label | 103.6 |
| | Claim | |
| Content Uniformity | Conforms to USP <905> | Conforms. |
| | | L1 = 14.9, Range |
| | | 91.2-111.4% |
| Residual Solvents by | <5000 PPM Acetic Acid | <300 PPM |
| Gas Chromatography | | |
| Water Content by KF | Not more than 12% | 8.8137% |
| Dissolution in Water | Visual Dissolution achieved | 20 seconds |
| for Injection Media | within 30 seconds | |
| Reconstituted Particle | Average Size not more | 1678 nm |
| Size quantification | than 4000 nm | |
| by Zetasizer | | |

Liquid Form (precursor to powder) Certificate of Analysis

| | | |
|---|---|---|
| Pre-lyophilization | Average Size Between | 860 nm |
| Particle Size | 500 and 2000 nm | |
| quantification by | | |
| Zetasizer | | |

As summarized in Table 2, in order to characterize the solubility with respect to pH, injectable chitosan powder comprising cisplatin was reconstituted within varying pH media and release of cisplatin was monitored at 355 nm in a UV-visible spectrometer.

TABLE 2
Relative solubility of injectable chitosan powder comprising
cisplatin in media of varying pH.

| | % Transmitted Light, |
|---|---|
| pH of media | λ = 355 nm |
| 5.5 | 100 |
| (Purified water, control) | |
| 2.00 | 89.5 |
| 2.93 | 89.8 |
| 4.05 | 89.7 |
| 5.06 | 90.2 |
| 5.90 | 89.2 |
| 7.08 | 90.5 |
| 7.92 | 91.0 |

In accordance with a first set of representative embodiments of the invention, a system for injectable local delivery of a therapeutic agent to a site in tissue is provided. The system includes polymeric matrix capable of forming a gel, i.e. a polymeric web like structure, in a solvent that contributes to keeping the embedded particles localized within the tissue. The gel matrix is formed by a composition including chitosan, a hydration promoter, a microparticle adhesion inhibitor, and a microparticle aggregation inhibitor. A plurality of microparticles are embedded within the gel matrix. The gel matrix is configured to open up to allow the drug loaded particles to have a limited range of motion within in the tissue. The microparticles contain a therapeutic agent and have a coating around the therapeutic agent. The coating of the microparticles includes chitosan so as to provide controlled release of the agent from the microparticles. Optionally, the hydration promoter is selected from the group consisting of ethylene glycol, propylene glycol, betapropylene glycol, glycerol and combinations thereof. Also optionally, the microparticle adhesion inhibitor is a non-ionic polymer, and, as a further option, the non-ionic polymer is HPMC or poloxamer. Additionally, as an option, the microparticle aggregation inhibitor is selected from the group consisting of monosaccharides, disaccharides, sugar alcohols, chlorinated monosaccharides, chlorinated disaccharides, and combinations thereof. Also optionally, the microparticles further include sodium tripolyphosphate. Optionally, in the system there is a free quantity of the therapeutic agent, embedded directly in the matrix, and not otherwise coated with chitosan, wherein the free quantity of the therapeutic agent constitutes between 20-80% of a total quantity of therapeutic agent in the system. Optionally, the chitosan in the matrix and the chitosan in the microparticles is pure chitosan. As a further option, the average diameter of the microparticles is from about 500 nm to about 2000 nm.

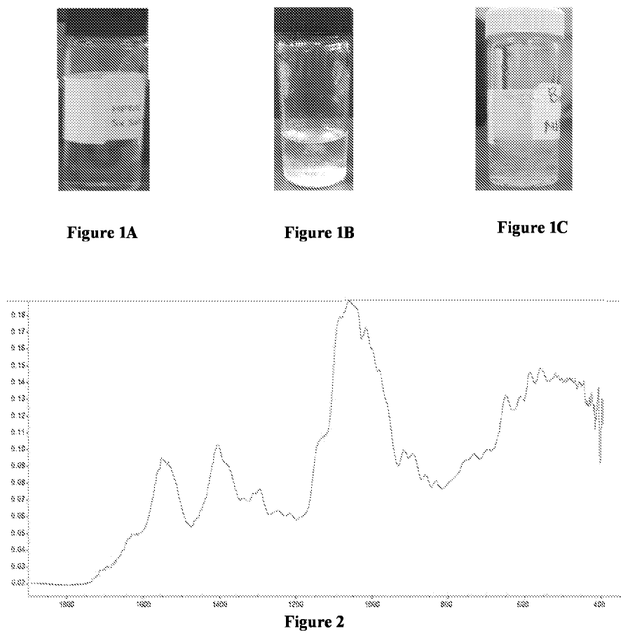

The matrix is configured to provide controlled release of the microparticles through the tissue. This system can be used to inject potent drugs with significant systemic toxic side effects, in a local manner to the damaged tissue such as cancerous tumor. The method for producing the invention further includes freezing the mixture at −80° C., to form a frozen layer precursor. Finally, the method for producing the invention includes drying the frozen layer precursor, to form a powder that, upon hydration, forms a gel matrix with microparticles embedded within the matrix. In some embodiments of the invention, the final product (a powder for reconstitution) is stable for over 6 months, but only if it is stored with a desiccant, heat-sealed within a water tight mylar foil pouch and stored in a 2-8° C. refrigerator. If these conditions are met, the system (sample PRV311, which includes the chitosan nanoparticles within a mesh) can be reconstituted into either a clear solution, or a heterogeneous microparticle suspension. PRV311's solubility is further increased due to chain fragmentation hydrolysis (for example, freeze-thaw hydrolysis) occurring in the mesh when frozen and in chitosan when PRV311 is gamma irradiated for use in patients. Properly stored PRV311 is shown in FIG. 1A, which is a clear solution/microparticle suspension.

As shown in FIG. 1B, PRV311 does not adequately reconstitute if it is stored at room temperature for several days prior to reconstitution. The vial in FIG. 1B was stored for five days at room temperature prior to reconstitution, and it forms a heterogeneous coarse suspension, unfit for injection. The vial in FIG. 1C shows another formulation, PRV111, frozen more quickly than PRV311, by means of liquid nitrogen, and also stored at room temperature for five days. The PRV111 formulation thus treated also forms a heterogeneous coarse suspension, unfit for injection.

Without being bound by theory, it is hypothesized that:
a) even with aggregation inhibitor added, particle-particle conformational aggregation occurs slowly (driven by van der Waals forces) in the powder formulations post lyophilization, but when powder formulations are stored in 2-8° C. conditions, the low temperature lends the system to greater kinetic stability;
b) some temperature-related factor causes a part of the mesh to become insoluble.

PRV311 is made by dispensing the liquid form of product into a vial, and letting the vial freeze over at least 8 hours in a −80° C. ambient environment. Following reconstitution in at least 1 mL of media, the appropriate volume of PRV311 is withdrawn with a Luer Lock Syringe. PRV311 is injected directly into the tumor using a needle gauge between 18 and 30. Each vial of PRV311 can contain between 0.1 and 100 mg of drug. Limits of dosage are dependent on the solubility of the encapsulated immunotherapy or small molecule in water. Frequency of administration depends on the site of treatment, the indication and the administrator's discretion.

In some embodiments of the invention, there is a water-soluble polymeric matrix formed by a composition including chitosan, a hydration promoter, a microparticle adhesion inhibitor, and a microparticle aggregation inhibitor. In accordance with yet another set of representative embodiments of the invention, there is provided a method for manufacturing a therapeutic agent delivery system. The method includes forming a first mixture with a plurality of microparticles. The microparticles contain a therapeutic agent and have a coating around the therapeutic agent, the coating including chitosan. The method also includes forming a second mixture from ingredients including the first mixture, chitosan, a hydration promoter, a microparticle adhesion inhibitor, and a microparticle aggregation inhibitor. The method further includes freezing the second mixture in a bath containing an aqueous alcoholic solution at a temperature above the freezing temperature of the aqueous alcoholic solution and at most −80° C., to form a frozen layer precursor. Finally, the method includes drying the frozen layer precursor, to form a porous polymeric matrix with microparticles embedded within the matrix. Optionally, the bath further contains dry ice. Also, optionally, the alcohol of the aqueous alcoholic solution is ethanol. As a further option, the aqueous alcoholic solution is from about 90 wt % ethanol to about 99 wt % ethanol. Optionally, the method further includes applying a second layer precursor to the frozen layer precursor, to form a solid comprising a first layer and a second layer. Optionally, the second layer comprises a therapeutic agent. Also, optionally, the drying is under vacuum.

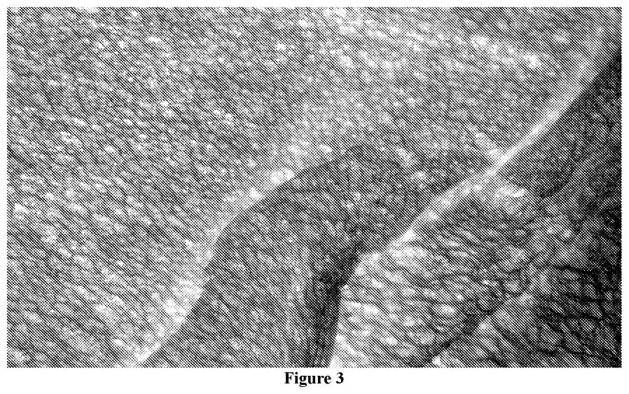

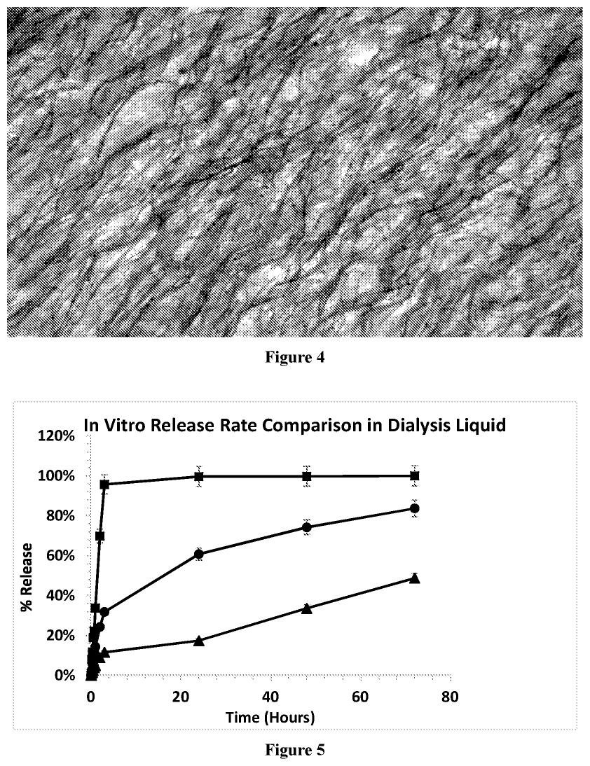

In some embodiments of the invention, the aqueous solution is frozen using a −80° C. ultra freezer. In other embodiments liquid nitrogen was used to freeze the solution. The freezing method using −80° C. Ultra freezer was compared to the liquid nitrogen freezing method and the results were surprising. As shown in FIG. 3, when frozen in liquid nitrogen (−196° C.) and lyophilized, a multilayered, dense, fabric-like structure is obtained. By contrast, as shown in FIG. 4, when frozen at −80° C., a more porous, uniform single layer polymer fiber is obtained. The structural differences between solutions resulting from the two methods had a major impact on the control and timing of the release of the encapsulated therapeutic. In certain embodiments comprising cisplatin, the inventors discovered through the course of product development that a specific combination of polymers and excipients is required in order for the cisplatin mesh to properly function. During development, permeation of cisplatin-containing nanoparticles from the mesh into tissue was hindered due to clumping and aggregation of the nanoparticles within the mesh. It was discovered that adding a combination of excipients and polymers resulted in total release of nanoparticles from the mesh. It was not immediately known why the inclusion of the combination results in ideal release and permeation; however upon microscopic analysis it was evident that its inclusion reacted with the nanoparticles and mesh structure in such a way that it formed microparticle “colonies” within the pores of the mesh structure (see FIG. 3). Unlike aggregation or clumping, which is typically one large mass and non-uniform, the “colonies” are near uniform in size and remain small enough to release from the mesh and permeate into the tissue. The structure of the mesh was also altered when the polymer excipient combination was included. The inclusion yielded a more crystalline structure with pores that were able to both hold and more easily release the microparticles. The aforementioned functionality attributed to the polymer excipient combination has been reported to be the result of the polymer's ability to open cell junctions within tissue; they initially were included in the composition of the mesh during testing for this purpose. However, its effect beyond altering cells had not been previously reported or observed, including both its effect on the structure of the mesh as well as its effect of “colonizing” and preventing aggregation of the microparticles. The effect of the polymers and excipients used in combination is synergistic, as their combined influence on release and penetration is far greater than the sum of their individual effects.

A release profile of particles at varying pHs is shown in FIG. 5. Powders were reconstituted in their respective media and placed inside of dialysis bags under stirring for 72 hours. Samples were taken and the percent release is shown, with microparticles at pH 6 (circles) releasing faster due to more rapid degradation, and those at pH 3 (triangles) released at a slower rate due to higher particle stability. Free cisplatin solution (squares) was used as a control. In this figure, it is apparent that lower pH slows the release of drug from microparticles.

According to some embodiments of the invention, the polymer excipient combination comprises chitosan, Hypromellose, and propylene glycol.

In some embodiments of the invention, the hydration promoter is selected from the group consisting of ethylene glycol, propylene glycol, beta-propylene glycol, glycerol and combinations thereof.

In some embodiments of the invention, the microparticle adhesion inhibitor is a non-ionic polymer.

In some embodiments of the invention, the non-ionic polymer is HPMC or Poloxamer.

In some embodiments of the invention, the microparticle aggregation inhibitor is selected from the group consisting of monosaccharides, disaccharides, sugar alcohols, chlorinated monosaccharides, chlorinated disaccharides, and combinations thereof.

In some embodiments of the invention, the microparticles further include sodium tripolyphosphate.

In some embodiments of the invention, the system further comprises a free quantity of the therapeutic agent, embedded directly in the matrix, and not otherwise coated with chitosan, wherein the free quantity of the therapeutic agent constitutes between 20-80% of a total quantity by weight of therapeutic agent in the system.

In some embodiments of the invention, the chitosan in the matrix and the chitosan in the microparticles is unmodified chitosan.

In some embodiments of the invention, the average diameter of the microparticles is from about 0.5 μm to about 2 μm.

In some embodiments of the invention, the therapeutic agent is an antibody such as an immunotherapeutic or small molecule such as a chemotherapeutic.

In some embodiments of the invention, the invention comprises a microparticle for targeted delivery of a therapeutic agent, the microparticle containing the unmodified therapeutic agent and unmodified chitosan.

In some embodiments of the invention, the microparticles are embedded within the matrix so as to be directly surrounded by, and in contact with, the matrix.

In some embodiments of the invention, there are provided systems for delivery of a therapeutic agent based on a polymeric matrix and microparticles which are improved by the addition of a hydration promoter to the matrix. Example hydration promoters include hygroscopic compounds such as glycols, for instance ethylene glycol, propylene glycol, beta-propylene glycol, and glycerol. Exemplary concentration ranges for the amount of hydration promoter include from about 0.001 to about 10 wt %, from about 0.01 to about 5 wt %, and from about 0.1 to about 1 wt %.

Without wishing to be bound to any particular theory, it is believed that the hydration promoter increases moisture absorption by the delivery system. This increase in hydration enables the rapid release and permeation of the microparticles from the matrix. It is also believed that the hydration promoter improves uniformity and durability by acting as a cryoprotectant during the manufacturing process of the delivery system. Again, without being bound to any particular theory, it is believed that the hydration promoter acts as a “spacer” between ice crystals and matrix polymer molecules, to ensure a uniform freezing pattern. The resulting structure is more flexible, uniform, and durable than in the absence of the hydration promoter.

In another set of representative embodiments, there are provided delivery devices improved by the addition of an adhesion inhibitor. Without wishing to be bound to any particular theory, it is believed that when the matrix and particles are made of materials bearing polar or ionically charged moieties, such as chitosan, the mobility of the particles suffers. In the instance of chitosan, it is believed that the interactions between acetyl and amine moieties of the polymer cause the particles to adhere to the matrix and inhibit their release.

It has been found that the inclusion of an adhesion inhibitor can mitigate adhesion of the matrix with the particles. Without being bound to any particular theory, it is believed that the adhesion inhibitor acts as a “spacer” between the chitosan of the particles and the chitosan in the body of the matrix, releasing the particles and allowing for improved drug release profiles. Representative example adhesion inhibitors include non-ionic polymers such as hydroxypropyl methylcellulose (HPMC). Depending on the application, the molar mass of the non-ionic polymer may be from about 1 kDa to about 200,000 kDa, while its viscosity may vary from about 10 cps to 100,000 cps. In representative embodiments, the molar mass of the non-ionic polymer is from about 10 kDa to 30 kDa, and its viscosity from about 10 cps to about 100 cps. Depending on the application, the amount of adhesion inhibitor may be from about 0.1 wt % to about 99 wt %. In some embodiments, the amount of adhesion inhibitor is from about 0.1 wt % to about 25 wt %.

In a further set of representative embodiments, delivery devices improved by the addition of an aggregation inhibitor are disclosed. Processes for manufacturing the delivery devices include freezing steps during which ice crystals may form within the matrix. Such crystals can force the microparticles into each other, creating particle aggregates where the particles are damaged or destroyed. Again without wishing to be bound to any particular theory, it is believed that aggregation inhibitors exert a cryoprotectant action by forming crystal microstructures which prevent aggregation of the particles. Carbohydrates and carbohydrate derivatives provide exemplary types of aggregation inhibitors, including monosaccharides, disaccharides, sugar alcohols, chlorinated monosaccharides, and chlorinated disaccharides such as sucralose. Depending on the application, the amount of aggregation inhibitor in the patch may be in the range from about 0.1 to about 50 wt %. In some embodiments, the amount of aggregation inhibitor is from about 1 to about 10 wt %.

In another set of representative embodiments, improved pure chitosan microparticles are provided. Traditional chitosan particles are manufactured with salts of chitosan characterized by a high degree of deacetylation and bearing electrically charged moieties, for example chitosan chloride and chitosan glutamate. It has been found that better results are provided if the particles are made from pure chitosan, a material characterized by not being a salt, that is, with its amine groups unprotonated, and having a degree of deacetylation of at least 70%. In particular, the particles are characterized by larger diameters than traditional particles. In some embodiments, the average diameter of the pure chitosan particles may range from about 200 to about 2000 nanometers. In other embodiments, the average diameter ranges from about 500 to about 2000 nanometers, and in additional embodiments from 500 to 1000 nm. [0043] In a further improvement, chitosan microparticles improved by the addition of sodium tripolyphosphate (STPP) are provided. Without wishing to be bound to any particular theory, it is believed that the STPP functions as a cross-linker to form the particles by acting as a negative counter-ion to the positively charged amine groups on chitosan. This electrostatic interaction forms ionic bonds that support the structure of the particles. Also without wishing to be bound to any particular theory, it is believed that the presence of sodium as positive counterion renders STPP a more effective crosslinker than other TPP salts.

It has also been found that when the gel matrix includes a free quantity of the therapeutic agent, embedded directly in the matrix and not otherwise coated with chitosan in the particles, the device is therapeutically more effective than comparable matrices which include either only a free quantity of the therapeutic agent or only therapeutic agent coated with chitosan. In representative embodiments, the free quantity of the therapeutic agent constitutes between 20-80% of the total quantity of therapeutic agent in the delivery system.

### EXAMPLES

The injectable, which can be reconstituted with common medias such as Water for Injection USP, 0.12% Saline USP, and 0.9% saline USP was tested in:

### Example 1: In-Vivo, Mice Study

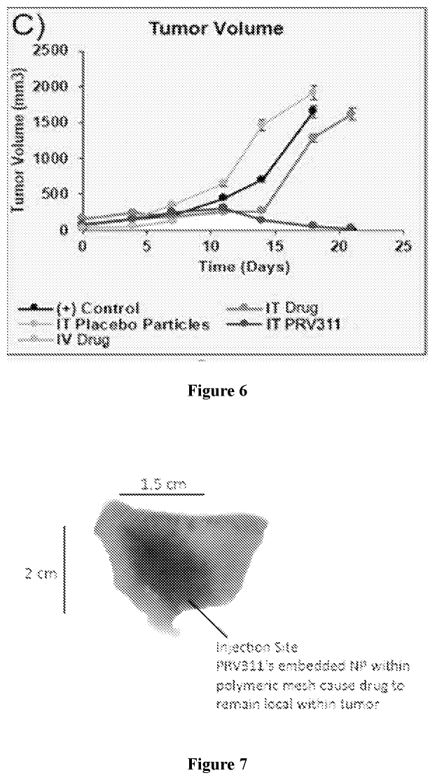

As shown in FIG. 6, intratumoral injection with PRV311 of mice grafted with cancer cells eliminated substantial tumor growth (blue curve). Here, the PRV311 composition included microparticles loaded with the anti-tumor cytokine IL-12. In comparison experiments, injections made into the tumor of a control saline solution (black curve), placebo particles (gray curve) showed minimal effect on tumor growth. Similarly, intravenous injection of IL-12 alone (green curve) showed no significant change in tumor growth rate when compared to control. Notably, intratumoral injection with IL-12 alone (red curve) merely delayed tumor growth, in contrast to the near elimination of tumor growth with IL-12 loaded PRV311 (blue curve). It is hypothesized that the injected PRV311's polymeric mesh causes microparticles containing PRV311 to be retained locally, increasing efficacy.

### Example 2: Ex-Vivo Tongue Study

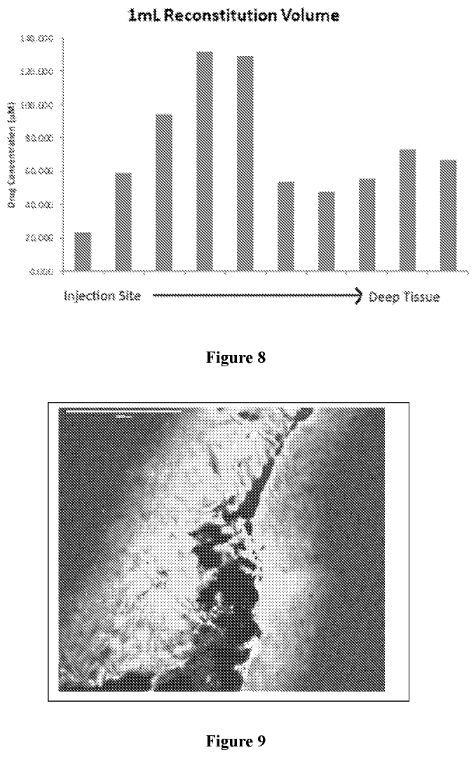

Pig tongue was injected with 500 μL of PRV311 using a 23G hypodermic luer-lock needle. Drug remained local due to the polymeric mesh. As shown in FIG. 8, drug concentration remained approximately uniform per cross section of tissue, and formed a bell curve like shape (resembling a sphere of injection).

### Example 3: Cow Brain

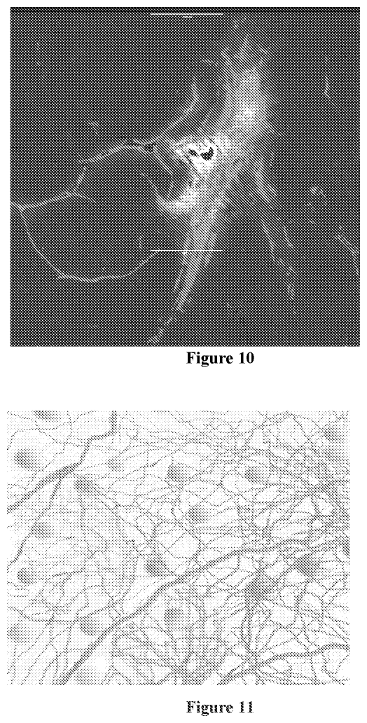

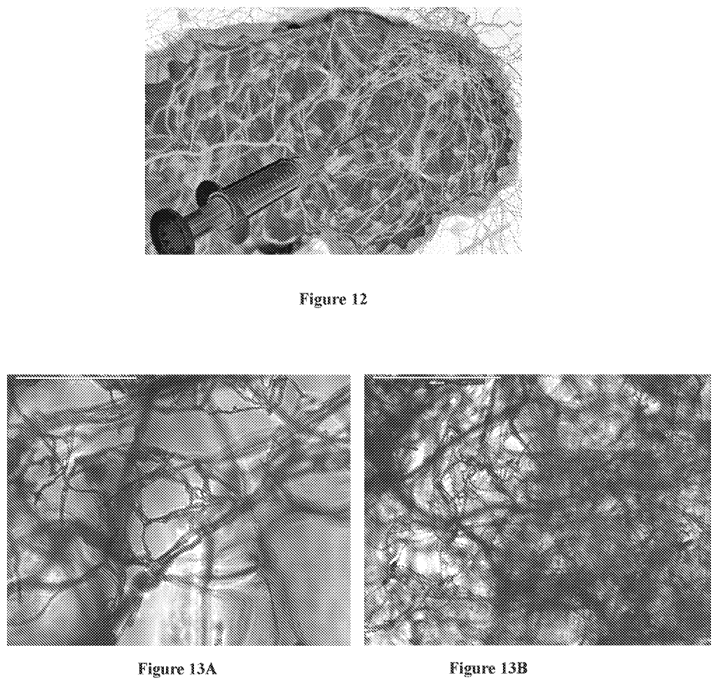

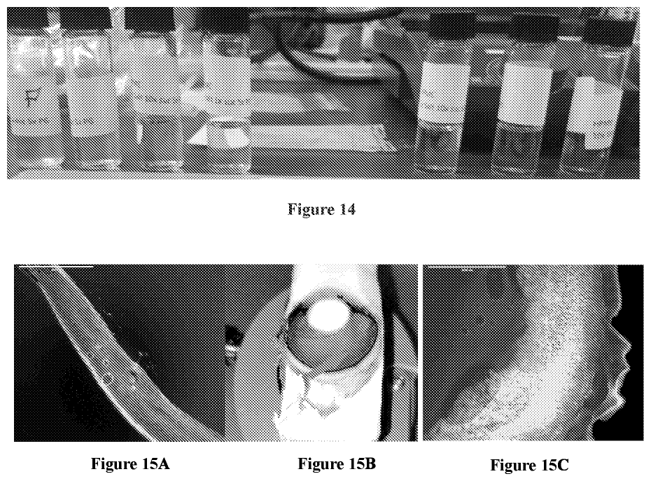

PRV311 (200 μL) was injected into a cow brain with a 26 Gauge needle. Tissue was sectioned for imaging via microscopy, as shown in FIG. 10.

Approximate dimensions of spread were 9 mm height×5 mm length for the volume injected. The red seen in FIG. 10 is Cy5 fluorophore dye attached to the Chitosan polymer, which shows the movement of the microparticles and mesh. The green is an encapsulated fluorophore that models the spread of the encapsulated drug. The mesh in the injectable keeps the fluorophore local and concentrated.

### Example 4: Drug Delivery into Sheep Corneas

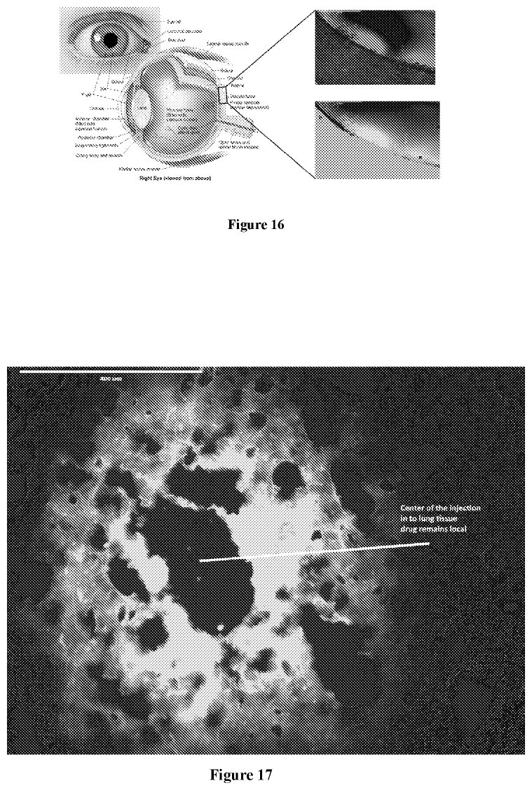

Studies performed in sheep have shown that PRV311 can deliver drug through the cornea into the intravitreal fluid (FIG. 15A, FIG. 15B, and FIG. 15C). PRV311 was injected into the vitreous body of the eye about 5 mm below the iris. The red image in the upper right of FIG. 16 shows how Cy5 labeled chitosan completely permeates the retina, choroid, and sclera. The green image in the bottom right of FIG. 16 shows the similar distribution of FITC labeled free drug during the same injection.

This supports PRV311 in offering an alternative to existing therapies for AMD. Data gathered to date shows:
Delivery of labelled particles into the cornea, and intravitreal fluid
Sustained release of bevacizumab over 4 months
Encapsulation enhances the stability and absorption of bevacizumab

### Example 5: Drug Delivery into Sheep Lung

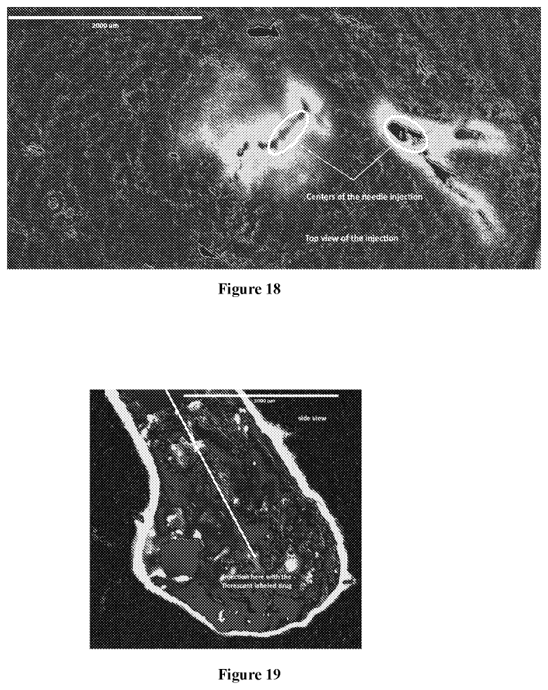

Studies performed in sheep have shown that PRV311 can deliver drug into lung tissue, where it remains localized (FIG. 17). The drug was held in high concentration of over 24 hours post injection before being frozen to prepare for sectioning.

### Example 6: Drug Delivery into Sheep Liver

As shown in FIGS. 17 and 18, PRV311 can deliver drug into liver tissue, where it remains localized. The drug was held in high concentration of over 24 hours post injection before being frozen to prepare for sectioning. As seen from the side-view in FIG. 19, the tissue type impacts the drug distribution pattern.

### Example 7: Drug Delivery into Sheep Pancreas

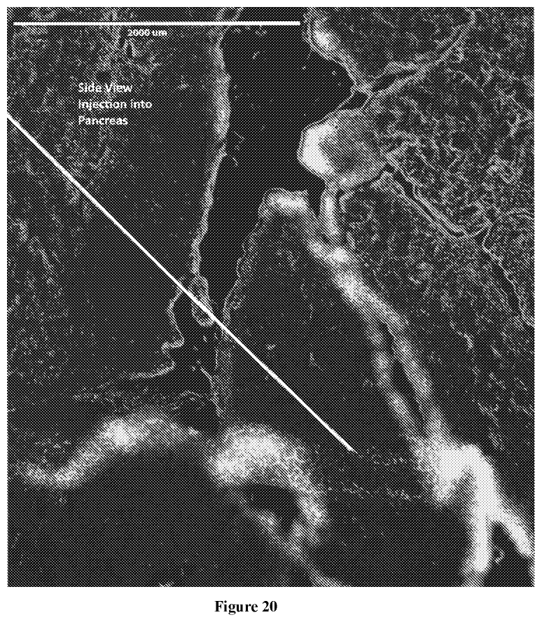

As shown in FIG. 20, PRV311 can deliver drug into pancreatic tissue, where it remains localized. The drug was held in high concentration of over 24 hours post injection before being frozen to prepare for sectioning.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A system for delivering a therapeutic treatment to a targeted population of cells of a subject, the system comprising:
a vial enclosed with a septum that is penetrable by a needle of a syringe to be used for administration of the therapeutic treatment;
a therapeutic composition disposed in the vial, the therapeutic composition provided for use in administration of the therapeutic treatment and comprising an aqueous solution including a chitosan gel and a plurality of particles embedded in the gel, the gel having a viscosity rendering it suitable for administration by injection;
the particles containing a therapeutic agent and having a coating around the therapeutic agent; and
the coating including chitosan so as to provide controlled release of the agent from the particles.

2. A system according to claim 1, wherein the aqueous solution further includes a compound selected from the group consisting of a hydration promotor, a particle adhesion inhibitor, a particle aggregation inhibitor, and combinations thereof, wherein:
(a) the hydration promotor is selected from the group consisting of ethylene glycol, propylene glycol, beta-propylene glycol, glycerol and combinations thereof,
(b) the particle adhesion inhibitor is selected from the group consisting of HPMC, poloxamer, and combinations thereof, and
(c) the particle aggregation inhibitor is selected from the group consisting of monosaccharides, disaccharides, sugar alcohols, chlorinated monosaccharides, chlorinated disaccharides, and combinations thereof.

3. A system according to any of claims 1 and 2, wherein the aqueous solution further includes sodium tripolyphosphate.

4. A system according to any of claims 1 and 2, wherein the particles are microparticles having an average diameter between 200 nm and 2000 nm.

5. A system according to any of claims 1 and 2, wherein the particles are microparticles having an average diameter between 500 nm and 2000 nm.

6. A system according to any of claims 1 to 5, the aqueous solution further including a free quantity of the therapeutic agent, not coated with chitosan, wherein the free quantity of therapeutic agent comprises between about 20% to about 80% of a total quantity by weight of therapeutic agent in the aqueous solution.

7. A system according to any of claims 1 to 6, wherein the therapeutic agent is an immunotherapeutic.

8. A system according to claim 7, wherein the immunotherapeutic is selected from the group consisting of an antibody, a cytokine, a small molecule immunotherapeutic, and combinations thereof.

9. A system according to any of claims 1 to 6, wherein the therapeutic agent is a chemotherapeutic.

10. A system according to claim 1, wherein the particles are in direct physical contact with the chitosan gel.

11. A method for treatment of a targeted population of cells of a subject, the method comprising:
obtaining the system according to claim 1;
loading the aqueous solution into the syringe;
injecting the aqueous solution into the targeted population of cells by means of the syringe.

12. A method according to claim 11, wherein the targeted population of cells includes a tumor.

13. A method according to claim 11, wherein the targeted population of cells is tissue in an organ.

14. A method according to claim 13, wherein the organ is selected from the group consisting of eye, lung, pancreas, liver, kidney, brain, heart, thyroid, and pituitary.

15. A system for delivering a therapeutic treatment to a targeted population of cells of a subject, the system comprising:
a vial enclosed with a septum that is penetrable by a needle of a syringe to be used for administration of the therapeutic treatment;
a therapeutic composition disposed in the vial, the therapeutic composition provided for use in administration of the therapeutic treatment and comprising a lyophilized precursor formulated so that upon mixing with water, it dissolves to provide an aqueous solution including chitosan gel and a plurality of particles embedded in the gel, the gel having a viscosity rendering it suitable for administration by injection;
the particles containing a therapeutic agent and having a coating around the therapeutic agent; and
the coating including chitosan so as to provide controlled release of the agent from the particles.

16. A lyophilization method for providing a system for delivering a therapeutic treatment to a targeted population of cells of a subject, the method comprising:
forming an aqueous solution including a chitosan gel and a plurality of particles, the particles containing a therapeutic agent and having a coating around the therapeutic agent, the coating including chitosan so as to provide controlled release of the agent from the particles;
freezing the first aqueous solution in a bath containing an aqueous alcoholic solution at a temperature above the freezing temperature of the aqueous alcoholic solution and at most −80° C., to form a frozen layer precursor;
drying the frozen layer precursor, to form anhydrous powder embedded with particles;
including the anhydrous powder in a vial enclosed with a septum that is penetrable by a needle of a syringe to be used for administration of the therapeutic treatment; and
adding water to the container to dissolve the anhydrous powder.

17. A lyophilization method according to claim 16, wherein the anhydrous powder further includes a compound selected from the group consisting of a hydration promoter, a particle adhesion inhibitor, and a particle aggregation inhibitor, and combinations thereof,
wherein the hydration promoter is selected from the group consisting of ethylene glycol, propylene glycol, beta-propylene glycol, glycerol and combinations thereof,
wherein the particle adhesion inhibitor is selected from the group consisting of hydroxypropylmethylcellulose, poloxamer, and combinations thereof, and
wherein the particle aggregation inhibitor is selected from the group consisting of monosaccharides, disaccharides, sugar alcohols, chlorinated monosaccharides, chlorinated disaccharides, and combinations thereof.

18. A method according to claim 16, wherein the anhydrous powder further includes sodium tripolyphosphate.

19. A method according to claim 16, wherein the particles are microparticles having an average diameter between 200 nm and 2000 nm.

20. A method according to claim 16, wherein the particles are microparticles having an average diameter between 500 nm and 2000 nm.

21. A system according to claim 1, configured for delivering a therapeutic treatment for an ocular condition by intravitreal injection.

22. A system according to claim 21, wherein the therapeutic agent is selected from the group consisting of an antibody, a cytokine, a small molecule immunotherapeutic, a chemotherapeutic, an aptamer, and combinations thereof.

23. A system according to claim 21 wherein the ocular condition is age-related macular degeneration.

24. A system according to claim 23, wherein the therapeutic agent is bevacizumab.
